(19) United States
(12) Patent Application Publication
(10) Pub. No.: US 2013/0012429 A1
(43) Pub. Date: Jan. 10, 2013

(54) ANTI-VIRAL THERAPY

(76) Inventors: Lucy Ann Eddowes, Knutsford (GB); Narayan Ramamurthy, Oxford (GB); Paul Klenerman, Oxford (GB); Alexander Hal Drakesmith, Oxford (GB)

(21) Appl. No.: 13/176,994

(22) Filed: Jul. 6, 2011

(51) Int. Cl. A61K 38/18; A61P 31/14; C12Q 1/02; C12N 7/06; C12Q 1/70; C12Q 1/68

(52) U.S. Cl. 514/3.7; 435/5; 435/6.1; 435/29; 435/238

(57) ABSTRACT

This invention relates to a method of treating viral infections, and compounds for use in the treatment of viral infections by modulating the BMP/SMAD signalling pathway. In particular, it relates to methods and compounds for treating hepatitis C virus infection and/or influenza virus infection. It also relates to methods for identifying compounds that are useful in the treatment of viral infections, in particular hepatitis C virus infection and/or influenza virus infection.

### CROSS REFERENCE TO RELATED APPLICATIONS

Not Applicable.

### STATEMENT REGARDING FEDERALLY FUNDED RESEARCH OR DEVELOPMENT

Not Applicable.

### FIELD OF INVENTION

This invention is generally directed to treatment viral infections. In particular, this invention is directed to methods and compounds for treating hepatitis C virus infection and/or influenza virus infection.

### BACKGROUND OF THE INVENTION

Viral infections are extremely widespread and cause a range of symptoms. There are relatively few effective treatments to reduce or prevent replication of viruses in cells of the body and therefore help the body to fight viral infections. Because of this, if a person is unable to clear a virus from the body it can result in a chronic infection with that virus. The lack of suitable anti-viral therapies makes viruses difficult to treat.

An example of a virus that affects a large number of people is hepatitis C virus (HCV). HCV infects around 3% of the word's population, around 200 million people. A small proportion of those infected spontaneously clear HCV, but for the rest, chronic infection significantly increases the risk of developing liver disease. The current treatment for chronic HCV infection is regular dosages for many weeks of interferons and ribavirin. This therapy is effective in the majority of cases, but a large number of patients still fail to clear the infection. Furthermore the drug regime also causes unpleasant side-effects. Chronic HCV is a global disease of both the developed and the developing world, and new medications are required to address the needs of different clinical settings.

Another example of a virus that affects a large number of people is influenza virus. Typically, in a year's normal two flu seasons (one per hemisphere), there are between three and five million cases of severe illness and up to 500,000 deaths worldwide. Although the incidence of influenza can vary widely between years approximately 36,000 deaths and more than 200,000 hospitalizations are directly associated with influenza every year in the United States alone. It is therefore of great interest to provide alternative treatments for influenza virus infection that can lessen the severity and/or the duration of the disease.

There is a need to improve treatment of viral infections, such as from HCV and influenza virus. Potential new therapies and identification of new therapeutic agents is required.

### SUMMARY OF THE INVENTION

A first aspect of the present invention is directed to a method of treatment or prevention of a viral infection in a subject comprising administering to said subject an effective amount of a compound that is a modulator of the activity of at least one component of the BMP/SMAD signalling pathway. In some embodiments of the first aspect, the viral infection is an infection with a virus selected from hepatitis C virus (HCV), hepatitis B virus, influenza virus, HIV-1, HIV-2, respiratory syncytial virus (RSV) and vaccinia virus. Preferably, the viral infection is an infection with HCV or influenza virus.

In some embodiments of the first aspect, the compound administered to the subject is an agonist of at least one component of the BMP/SMAD signalling pathway. In other embodiments, the compound is an antagonist of at least one component of the BMP/SMAD signalling pathway.

In some embodiments of the first aspect, the compound administered to the subject increases the activity of the BMP/SMAD signalling pathway in the subject's cells. In other embodiments, the compound decreases the activity of the BMP/SMAD signalling pathway in the subject's cells.

In some embodiments of the first aspect, the compound administered to the subject modulates the activity of the BMP/SMAD signalling pathway in the subject's cells towards the activity expected in non virus infected cells. In some embodiments, the compound increases the activity of the BMP/SMAD signalling pathway in the subject's cells to a higher activity than expected in non virus-infected cells. In other embodiments, the compound decreases the activity of the BMP/SMAD signalling pathway in the subject's cells to a lower activity than expected in non virus-infected cells.

In a second aspect, the invention is directed to a method for identifying a compound that is useful in the treatment of infection with a virus. Such a method includes the steps of selecting a compound that modulates the activity of an intermediate in the BMP/SMAD signalling pathway, and testing whether the compound reduces or prevents replication of said virus in virus-infected cells in vitro.

In some embodiments of the second aspect of the invention, the method further includes the step of making a quantity of the selected compound. In some preferred embodiments of the second aspect of the invention, the virus is HCV or influenza virus.

A third aspect of the present invention is directed to a method for obtaining an indication helpful in the assessment of whether viral infection in an individual will respond to treatment with antiviral treatment. Such a method includes the steps of providing a sample of cells or a body fluid from the individual, measuring the level in the cells or the body fluid of at least one indicator selected from: HAMP mRNA (which encodes hepcidin), ID1 mRNA, HJV mRNA, SMAD6 mRNA, and SMAD7 mRNA, hepcidin, hemojuvelin, SMAD6 protein, SMAD7 protein, and comparing the level of the at least one indicator in the cells or the body fluid with the level of the same indicator expected in control cells or body fluid that is not infected with the virus.

In some embodiments of the third aspect, the viral infection is HCV infection and the cells have reduced HAMP mRNA, reduced HJV mRNA and/or reduced levels of ID1 mRNA compared with control cells that are not infected with HCV.

In other embodiments of the third aspect, the infection is with HCV and the cells have increased SMAD6 and/or increased SMAD7 compared to control cells that are not infected with HCV.

In yet other embodiments of the third aspect, the infection is an infection with HCV and SMAD6 and SMAD7 are relatively increased compared to ID1 in the cells and/or SMAD7 is relatively increased compared to HAMP in the cells.

In some preferred embodiments of the third aspect, the sample is a sample of liver cells and the virus is HCV. In other preferred embodiments, the body fluid is blood and the at least one indicator is hepcidin.

In still other embodiments of the third aspect, the virus is HCV and the antiviral treatment is treatment with interferon and/or ribavirin.

In a fourth aspect, the present invention is directed to a method for inhibiting viral replication comprising modulating the activity of a component of the BMP/SMAD signalling pathway. In a preferred embodiment of this aspect, viral replication is replication of HCV or influenza virus.

These and other features and advantages of various exemplary embodiments of the methods according to this invention are described, or are apparent from, the following detailed description of various exemplary embodiments of the methods according to this invention.

### BRIEF DESCRIPTION OF DRAWINGS

There now follows by way of example only a detailed description of the present invention with reference to the accompanying drawings, in which;

FIG. 1 shows a diagram of the BMP/SMAD signalling pathway in hepatocytes. TMPRSS6 inhibits the BMP/SMAD signalling pathway. BMPs, (exemplified by BMP6), bind Type I/II receptors and a co-receptor, which for BMP6 is HJV, leading to the phosphorylation of the Type I receptor by the Type II receptor. The Type I receptor phosphorylates one of several receptor-SMADs; SMAD1, SMAD5 or SMAD8. These bind SMAD4 and the complex translocates to the nucleus where it binds BMP response elements in promoters of BMP target genes and activates gene transcription. In liver cells, BMP target genes include hepcidin and ID1. Two other genes targeted are SMAD6 and SMAD7—the encoded proteins mediate negative feedback onto the BMP/SMAD signaling pathway. In addition the membrane expressed serine protease TMPRSS6 inhibits BMP6 signalling.

FIG. 2 shows the results of an experiment in which Influenza virus infected HuH7 cells were mixed 1:10 with uninfected HuH7 cells and then cultured for 24 hours with no added treatment, or with interferon alpha and interferon beta, or with BMP6, or with BMP6 and interferons alpha and beta. The cells were then stained for surface expression of the viral protein haemagglutinin. BMP6 restricts spread of the Influenza virus (as indicated by a reduced level of haemagglutinin measured by flow cytometric analysis of cells) better than interferon alpha and beta (note log scale).

FIG. 3 shows the expression of BMP/SMAD signalling pathway genes in HCV patient liver biopsies.

a HAMP mRNA levels, quantified by qRT-PCR relative to the endogenous control gene GAPDH, were significantly decreased in HCV liver biopsies (n=57) compared to control liver biopsies (n=8) (p=0.0027; Mann-Whitney t-test);
b there was no alteration in HJV mRNA levels;
c SMAD6 mRNA was significantly increased (p=0.0080) (both Mann-Whitney t-test);
d mean SMAD7 levels were increased but not significantly (unpaired t-test).
(e-h) Patients with known treatment outcome (n=26) were divided by outcome (SVR and NR) and compared to the control group (n=8).
e-g HAMP mRNA expression was reduced in both groups compared to controls (P<0.0001) whereas HJV and ID1 (n=17) were significantly reduced only in the NR group (HJV: P=0.0027; ID1 P=0.0515).
h Levels of BMP target genes ID1 and HAMP mRNA levels were proportional to each other in all three groups.
i, j The expression of SMAD6 and SMAD7 was compared to the expression of the BMP-regulated genes ID1 and HAMP by dividing the expression of the I-SMAD by the expression of ID1 or HAMP. Compared to the expression of ID1 the expression of SMAD6 and SMAD7 was relatively increased in the NR group (SMAD6: P=0.0032; SMAD7: P=0.0023). * indicates p<0.05, ** indicates p<0.01

FIG. 4 shows how I-SMAD expression relative to HAMP and HJV is increased in non-responders
a, b SMAD7 mRNA (but not SMAD6 mRNA) was significantly increased relative to HAMP mRNA expression in the NR group (SMAD6: P=0.0932; SMAD7: P=0.0727; One-way ANOVA, Bonferroni's multiple comparison test; * indicates p<0.05, ** indicates p<0.01).
c, d SMAD6 and SMAD7 mRNA was significantly increased relative to HJV mRNA expression in the NR group (SMAD6/HJV: P=0.1218; SMAD7/HJV: P=0.0186).

FIG. 5 shows the effects of HCV infection and TNF-alpha on BMP signalling in vitro
a HJV mRNA levels, quantified by qRT-PCR relative to GAPDH expression, declined in HCV-infected HuH7.5 cells with increasing MOI (n=3); b SMAD6 and SMAD7 mRNA levels increased with multiplicity of infection (n=3). c Uninfected or HCV-infected (MOI=0.02) HuH7.5 cells were incubated with a titration of BMP6 overnight; HAMP mRNA induction in response to BMP6 was significantly suppressed in HCV-infected cells (p<0.01, paired two-tailed t-test; n=6). d TNFA levels increased with multiplicity of infection (n=3). e, f Incubation of Hep3B cells with exogenous TNF-alpha results in the loss of HJV expression and increased SMAD6 and SMAD7 expression (n=4). g Hep3B cells transfected with siRNA targeting SMAD6, SMAD7 or both genes have increased HAMP expression, which reaches significance only when both genes are targeted (P<0.0001; n=3). h, i Knock-down of SMAD7 increases SMAD6 mRNA levels (P<0.0001; n=3) and vice-versa (P<0.0001; n=3). g-i One-way ANOVA with Bonferroni's Multiple Comparison Test; * indicates p<0.05, ** indicates p<0.01.

FIG. 6 shows how HAMP mRNA correlates with hepcidin peptide
Hep3B cells were incubated with increasing concentrations of BMP9 and IL6 at 50 ng/ml to induce hepcidin expression. HAMP mRNA was measured by qRT-PCR relative to the endogenous control GAPDH and hepcidin peptide in the culture supernatants was measured by ELISA (see supplementary methods). qRT-PCR and ELISA are proportional to each other except at the lower levels of expression where peptide detection sensitivity is below the sensitivity of qRT-PCR. qRT-PCR was the preferred method to accurately detect variations in hepcidin gene expression in this study.

FIG. 7 shows the effect of SMAD6 and SMAD7 knock-down on HAMP, SMAD6 and SMAD7 mRNA expression in HuH7 cells.
a Knock-down of SMAD6 or SMAD7 alone increases HAMP mRNA expression but not significantly, but knock-down of both strongly upregulates HAMP mRNA in HuH7 cells (P=0.0031; n=3). b, c Knock-down of SMAD7 increases SMAD6 mRNA expression and vice-versa (SMAD6: P=0.0014; SMAD7: P=0.0020; n=3). g-i One-way ANOVA with Bonferroni's Multiple Comparison Test.

FIG. 8 shows how Smad6 and Smad7 alter HAMP and ID1 mRNA expression.
a Hep3B cells transfected with plasmids encoding murine Smad6 and Smad7 have reduced HAMP and ID1 mRNA expression, particularly when the plasmids are cotransfected. b Murine Smad6 and Smad7 mRNAs are detectable after transfection.

FIG. 9 shows how TNF-alpha mediates the HCV-induced inhibition of BMP signalling
a TNF-alpha treated Hep3B cells show reduced p-SMAD1/5/8 after incubation with BMP6 for both 1 hr (n=2) and 18 hr (n=2). b, c Pre-treatment of Hep3B cells with TNF-alpha reduces their response to overnight incubation with BMP6 (Wilcoxon matched pairs t-test; n=3) and BMP9 (paired two-tailed t-test; n=3). d Neutralizing TNF-alpha over the course of a 10 day HCV infection in vitro restores BMP-mediated hepcidin induction (repeated measures non-Gaussian one-way ANOVA with Dunn's Multiple Comparison Test; n=3).

FIG. 10 shows the IL6 induction and signalling in the context of HCV and TNF-alpha.
a IL6 mRNA expression was not significantly altered in HuH7.5 cells infected for 8-10 days versus uninfected cells cultured alongside (p=0.3673; paired two-tailed t-test; n=7). b TNF-alpha pre-treatment suppresses the induction of HAMP mRNA in response to IL6.

FIG. 11 shows how exogenously added hepcidin does not alter HCV replication.
1 uM hepcidin peptide was added to HuH7.5 cell cultures in which HCV had been inoculated 3 days previously. After a further 7 days HCV RNA in supernatant or in cells was not significantly altered by hepcidin.

FIG. 12 shows how BMP6 has antiviral activity against HCV.
a The levels of HCV-RNA in the supernatants of HuH7.5 cells incubated with BMP6 at the stated doses is reduced in the cultures treated with BMP6. b The levels of HCV-RNA in the supernatants at 5 days post-infection is significantly lower in those cultures treated with BMP6 for the duration of the infection. Overnight incubation of the cultures with IFNa also results in a significant reduction in supernatant HCV-RNA (P=0.0013; One-way ANOVA with Bonferroni's Multiple Comparison Test n=3). c, d 10 days post-infection the levels of supernatant and cellular HCV-RNA is lower in cells treated with 18 nM BMP6 for 7 days or IFNa overnight (supernatants: P=0.0197; cellular: P=0.0208; Repeated measures one-way ANOVA with Bonferroni's Multiple Comparison Test; n=3).

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

It is an aim of the present invention to provide new treatments for viral infections, in particular treatments for HCV and influenza virus infection by modulating the activity of the BMP/SMAD signalling pathway.

According to a first aspect the invention provides a method of treatment or prevention of a viral infection comprising the administration of a compound that is a modulator of the activity of at least one component of the BMP/SMAD signalling pathway.

It is advantageous to modulate the activity of the BMP/SMAD signalling pathway because infection of cells with viruses changes the activity of the BMP/SMAD signalling pathway to make the conditions inside the cell more favourable for viral replication. If the activity of the BMP/SMAD signalling pathway can be modulated or returned to the activity in an uninfected cell it makes the conditions in the cell less favourable for viral replication and therefore inhibits viral replication.

In one embodiment the viral infection may be infection with a virus selected from HCV, hepatitis B virus, influenza virus, HIV-1, HIV-2, respiratory syncytial virus (RSV) and vaccinia virus. In one embodiment the viral infection may be HCV, the infection may be acute or chronic. In another embodiment the viral infection may be infection with influenza virus.

In another embodiment the invention provides a compound for use in the prevention or treatment of a viral infection, wherein the compound is a modulator of the BMP/SMAD signalling pathway. The compound may, for example be a compound identified by a method of the present invention.

In one embodiment the invention provides a compound for use in the treatment of an acute or in the treatment of a chronic viral infection, wherein the compound is a modulator of the BMP/SMAD signalling pathway. The modulator may be an agonist of the BMP/SMAD signalling pathway. The modulator may be an antagonist of the BMP/SMAD signalling pathway.

In one embodiment the invention provides a compound for use in the prevention or treatment of a viral infection, wherein the compound is an agonist of the BMP/SMAD signalling pathway. For example, the viral infection may be infection with HCV or a virus that decreases the activity of the HCV signalling pathway.

In another embodiment the invention provides a compound for use in the prevention or treatment of a viral infection wherein the compound is an antagonist of the BMP/SMAD signalling pathway. For example the viral infection may be infection with influenza virus or a virus that increases the activity of the BMP/SMAD signalling pathway.

In another embodiment the invention provides a compound for use in the manufacture of a medicament for treatment or prevention of a viral infection wherein the compound is a modulator of at least one component of the BMP/SMAD signalling pathway. The compound may be an agonist of the BMP/SMAD signalling pathway and be useful in the treatment or prevention of an infection with a virus that decreases the activity of the BMP/SMAD signalling pathway, for example HCV infection, which results in reduced levels of hepcidin. An agonist of the BMP/SMAD signalling pathway may be a molecule that increases the activity of the BMP/SMAD signalling pathway. An agonist of the BMP/SMAD signalling pathway may be an agonist of a molecule that increases the activity of the BMP/SMAD signalling pathway or an antagonist of a molecule that reduces the activity of the BMP/SMAD signalling pathway.

The compound may be an antagonist of the BMP/SMAD signalling pathway and be useful in the treatment or prevention of an infection with a virus that increases the activity of the BMP/SMAD signalling pathway. An antagonist of the BMP/SMAD signalling pathway may be a molecule that decreases that activity of the BMP/SMAD signalling pathway. An agonist of the BMP/SMAD signalling pathway may be an antagonist of a molecule that increases the activity of the BMP/SMAD signalling pathway or an agonist of a molecule the reduces the activity of the BMP/SMAD signalling pathway.

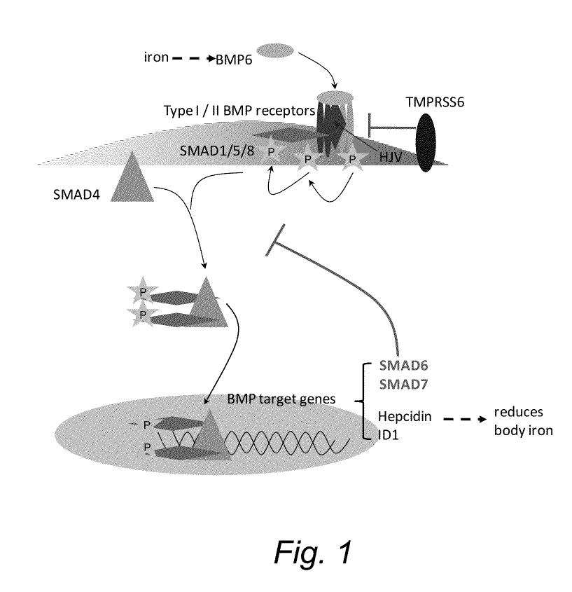

An example of the BMP/SMAD signalling pathway in liver cells is shown in FIG. 1. The compound may be a modulator of the activity or expression of any component of the BMP/SMAD signalling pathway in particular the compound may be a modulator of the activity or expression of Type I BMP receptors, the compound may be a modulator of the activity or expression of Type II BMP receptors, the compound may be a modulator of the activity or expression of hemojuvelin (HJV), the compound may be a modulator of the activity or expression of SMAD1, the compound may be a modulator of the activity or expression of SMAD4, the compound may be a modulator of the activity or expression of SMAD5, the compound may be a modulator of the activity or expression of SMAD6, the compound may be a modulator of the activity or expression of SMAD7, the compound may be a modulator of the activity or expression of SMAD8, the compound may be a modulator of the activity or expression of TMPRSS6 or the compound may be a modulator of the activity or expression of a BMP protein, in particular the compound may be a modulator of the activity or expression of BMP6. The compound may be a modulator of the expression of a BMP target gene such as hepcidin, or ID1. The compound may be a modulator of the amount or activity of an mRNA that encodes a component of the BMP/SMAD signalling pathway.

The compound may be a modulator of the activity or expression of any of the BMP type I and type II receptors in Table 1.

TABLE 1

| Official name | Gene ID | a.k.a. | What does it do? |
|---|---|---|---|
| ACVRL1 | 94 | HHT; ALK1; | Mutations in this |
| Activin A | | HHT2; ORW2; | gene are associated |
| receptor type II- | | SKR3; ALK-1; | with hemorrhagic |
| like 1 | | TSR-I; | telangiectasia type 2, |
| | | ACVRLK1; | also known as Rendu- |
| | | ACVRL1 | Osler-Weber |
| | | | syndrome 2. |
| ACVR1 | 90 | FOP; ALK2; | Mutations in this |
| Activin A | | SKR1; TSRI; | gene are associated |
| receptor, type I | | ACTRI; | with fibrodysplasia |
| | | ACVR1A; | ossificans |
| | | ACVRLK2; | progressive. |
| | | ACVR1 | |
| ACVR2A | 92 | ACVR2; | |
| Activin A | | ACTRII; | |
| receptor, | | ACVR2A | |
| type IIA | | | |
| ACVR2B | 93 | ACTRIIB; | This gene encodes |
| Activin A | | ActR-IIB; | activin A type IIB |
| receptor, type IIB | | MGC116908; | receptor, which |
| | | ACVR2B | displays a 3- to 4-fold |
| | | | higher affinity for the |
| | | | ligand than activin A |
| | | | type II receptor. |
| BMPR1A | 657 | ALK3; SKR5; | |
| Bone | | CD292; | |
| morphogenetic | | ACVRLK3; | |
| protein receptor, | | 10q23del; | |
| type 1A | | BMPR1A | |
| BMPR1B | 658 | ALK6; ALK-6; | Mutations in this |
| Bone | | CDw293; | gene have been |
| morphogenetic | | BMPR1B | associated with |
| protein receptor, | | | primary pulmonary |
| type 1B | | | hypertension. |
| BMPR2 | 659 | BMR2; PPH1; | Mutations in this |
| Bone | | BMPR3; BRK- | gene have been |
| morphogenetic | | 3; T-ALK; | associated with |
| protein receptor, | | BMPR-II; | primary pulmonary |
| type II | | FLJ41585; | hypertension, both |
| (serine/threonine | | FLJ76945; | familial and |
| kinase) | | BMPR2 | fenfluramine- |
| | | | associated, and with |
| | | | pulmonary |
| | | | venoocclusive |
| | | | disease. |
| ENG | 2022 | RP11-228B15.2, | Possibly a BMP9 |
| Endoglin | | CD105, END, | receptor. |
| | | FLJ41744, | |
| | | HHT1, ORW, | |
| | | ORW1 | |

The bone morphogenetic protein (BMP) receptors are a family of transmembrane serine/threonine kinases that include the type I receptors BMPR1A and BMPR1B and the type II receptor BMPR2. These receptors are also closely related to the activin receptors, ACVR1 and ACVR2. The ligands of these receptors are members of the TGF-beta superfamily. TGF-betas and activins transduce their signals through the formation of heteromeric complexes with 2 different types of serine (threonine) kinase receptors: type I receptors of about 50-55 kD and type II receptors of about 70-80 kD. Type II receptors bind ligands in the absence of type I receptors, but they require their respective type I receptors for signalling, whereas type I receptors require their respective type II receptors for ligand binding.

In one embodiment the compound may return the level or activity of a component of the BMP/SMAD signalling pathway in a treated cell to the level in a non-virus-infected cell. In another embodiment the compound may increase the level or the activity of a component of the BMP/SMAD signalling pathway compared to the level or the activity of that component in a virus-infected cell. In another embodiment the compound may increase the level or activity of a component of the BMP/SMAD signalling pathway compared to the level or the activity in a non virus-infected cell. In another embodiment the compound may decrease the level or the activity of a component of the BMP/SMAD signalling pathway compared to the level or activity of that component in a virus-infected cell or compared to the level or activity in a non virus-infected cell.

It is advantageous to modulate the activity of the BMP/SMAD signalling pathway in virus-infected cells because this can inhibit replication of the virus. Some viruses increase the activity of the BMP/SMAD signalling pathway and it can inhibit replication of these viruses if the activity of the BMP/SMAD signalling pathway is reduced to return the activity of the pathway to the level of activity in normal or a non virus-infected cell. It can also inhibit replication of these viruses if the activity of the BMP/SMAD signalling pathway is reduced below the level of activity in a normal or a non virus-infected cell. Some viruses decrease activity of the BMP/SMAD signalling pathway and it can inhibit replication of these viruses if the activity of the BMP/SMAD signalling pathway is increased to the level of activity in a normal or non virus-infected cell. It can also inhibit replication of these viruses if the activity of the BMP/SMAD signalling pathway is increased above the level of activity in a normal or a non virus-infected cell.

In one embodiment the compound may be a small molecule. In another embodiment the compound may be a polypeptide, the compound may be an antibody, the compound may be a DNA molecule, the compound may be an RNA molecule, the compound may be a short interfering RNA (siRNA).

In one embodiment the small molecule stimulates the activity of the BMP/SMAD signalling pathway by altering the phosphorylation of Type I or Type II BMP receptors.

A small molecule may be a molecule that is less than 800 daltons. In one embodiment a small molecule is not a biopolymer.

In another embodiment the small molecule modulates the activity of the BMP/SMAD signalling pathway by modulating the activity of TMPRSS6 (also called Matriptase-2). TMPRSS6 inhibits hepcidin activation by cleaving membrane hemojuvelin and TMPRSS6 decreases the activity of the BMP/SMAD signalling pathway. In one embodiment the small molecule may be an agonist of TMPRSS6 which has the effect of decreasing the activity of the BMP/SMAD signalling pathway. An agonist of TMPRSS6 may decrease the activity of the BMP/SMAD signalling pathway in a virus-infected cell to return the activity of the BMP/SMAD signalling pathway to the level expected in a non virus-infected cell. In another embodiment an agonist of TMPRSS6 may decrease the activity of the BMP/SMAD signalling pathway to a level lower than expected in a non virus-infected cell. In one embodiment the present invention provides an agonist of TMPRSS6 for use in the treatment of a viral infection that is treatable or preventable by decreasing the activity of the BMP/SMAD signalling pathway.

In one embodiment the small molecule may be an antagonist or inhibitor of TMPRSS6. As TMPRSS6 is a transmembrane serine protease that inhibits hepcidin expression by decreasing activity of the BMP/SMAD signaling pathway, an antagonist or inhibitor of TMPRSS6 has the effect of increasing the activity of the BMP/SMAD signalling pathway. An antagonist or inhibitor of the serine protease TMPRSS6 may increase the activity of the BMP/SMAD signalling pathway in a virus-infected cell to return the activity of the BMP/SMAD signalling pathway to the level expected in a non virus-infected cell. In another embodiment an antagonist of TMPRSS6 may increase the activity of the BMP/SMAD signalling pathway to a level higher than expected in a non virus-infected cell. An antagonist or inhibitor of TMPRSS6 may be used in the treatment of HCV infection. In one embodiment the present invention provides an antagonist or inhibitor of TMPRSS6 for use in the treatment of a viral infection that is treatable or preventable by increasing the activity of the BMP/SMAD signalling pathway. Preferably the viral infection is HCV infection. Preferably the antagonist or inhibitor is an antagonist or inhibitor of TMPRSS6, for example an inhibitor as described in Sisay et al. J Med Chem. 2010 Aug. 12; 53(15):5523-35. In one embodiment the inhibitor of the BMP/SMAD signalling pathway is a small molecule that inhibits the activity of BMP receptors, for example LDN-193189. LDN-193189 is described in Gregory et al. Bioorg Med Chem Lett. 2008 Aug. 1; 18(15): 4388-4392.

TMPRSS6 is a useful target for compounds that modulate the activity of the BMP/SMAD signalling pathway because expression of TMPRSS6 is restricted to the liver so that modulating the activity of this molecule can have an effect mostly on liver cells.

It is advantageous to use a small molecule to modulate the activity of the BMP/SMAD signalling pathway because they are easy and cheap to manufacture. Small molecules are often able to penetrate into the cells and are often suitable for oral administration.

In one embodiment the compound may be administered in addition to or synergistically with another therapy. For example, HCV infection may be treated with a compound that is a modulator of the BMP/SMAD signalling pathway according to the present invention in addition to or sequentially with interferon and/or ribavirin.

In one embodiment the compound may have an effect selected from decreasing the level of SMAD6 and/or increasing the level of hepcidin in cells infected with HCV.

In one embodiment the compound may be BMP6 or a BMP6 agonist.

In one embodiment the compound is not BMP7 or an agonist of BMP7 for the treatment of HCV infection.

In one embodiment the method inhibits replication of HCV virus and/or influenza virus.

According to a further aspect of the present invention we provide a method for inhibiting viral replication comprising modulating the activity of a component of the BMP/SMAD signalling pathway. In one embodiment the viral replication is replication of HCV or influenza virus.

The method may comprise use of a compound that modulates the BMP/SMAD signalling pathway, for example an agonist or an antagonist of the BMP/SMAD signalling pathway.

According to a further aspect the present invention provides a method for identifying a compound that is useful in the treatment of infection with a virus comprising the steps of:
selecting a compound that modulates the activity of an intermediate in the BMP/SMAD signalling pathway;
testing whether the compound reduces or prevents replication of said virus in vitro.

The method may further comprise the step of making a quantity of the selected compound.

The method may be used for identifying compounds useful in the treatment of HCV and/or influenza virus, preferably HCV.

The compound may modulate the level of any intermediate in the BMP/SMAD signalling pathway including the activity or expression of Type I BMP receptor, Type II BMP receptor, hemojuvelin TMPRSS6, SMAD1, SMAD4, SMAD5, SMAD6, SMAD7, SMAD8 or a BMP protein, in particular BMP6. The modulator may be a modulator of the expression of a BMP target gene such as hepcidin, or ID1. In one embodiment the modulator does not modulate the level of BMP7.

The compound may be an agonist or antagonist of any intermediate in the BMP/SMAD signalling pathway or may increase or decrease expression of any intermediate in the BMP/SMAD signalling pathway.

The compound may be an agonist that increases the level or activity of any intermediate in the BMP/SMAD signalling pathway that increases the activity of the pathway, such as increasing the level or activity of components of the pathway that lead to increased hepcidin production. For example, the selected compound may increase the level or activity of BMP6, HJV, SMAD4, ID1 and/or hepcidin and/or the mRNA that encodes them, or decrease the activity of TMPRSS6, SMAD6 or SMAD7 and/or the mRNA that encodes them.

An agonist may increase the overall level of activity of the BMP/SMAD signalling pathway resulting in increased hepcidin expression. An increase in the overall level of activity of the BMP/SMAD signalling pathway may be measured by measuring an increase in hepcidin expression. A rise in the overall activity of the BMP/SMAD signalling pathway may be measured by measuring an increase in expression of the BMP-regulated gene ID1. Although ID1 is not a member of the BMP/SMAD signalling pathway its expression correlates with the activity of the pathway and it is a good measure of the activity of the BMP/SMAD signalling pathway as a whole.

In one embodiment the compound may be BMP6 or an agonist of BMP6. In one embodiment the present invention provides BMP6 and/or an agonist of BMP6 for use in the treatment of a viral infection that is treatable or preventable by increasing the activity of the BMP/SMAD signalling pathway.

An agonist of BMP6 may increase the activity of the BMP/SMAD signalling pathway in a virus-infected cell to return the activity of the BMP/SMAD signalling pathway to the level expected in a non virus-infected cell. In another embodiment an agonist of BMP6 may increase the activity of the BMP/SMAD signalling pathway to a level higher than expected in a non virus-infected cell.

In one embodiment the compound may be a small molecule agonist or antagonist of one of the intermediates in the BMP/SMAD signalling pathway. In another embodiment the compound may be a nucleotide sequence that is an agonist or antagonist of expression of one of the intermediates in the BMP/SMAD signalling pathway.

The compound may be an antagonist that decreases the level or activity of any intermediate in the BMP/SMAD signalling pathway that decreases the activity of the pathway, such as decreasing the level or activity of components of the pathway that lead to decreased hepcidin production. For example the selected compound may decrease the level or activity of TMPRSS6, SMAD6, SMAD7 or the mRNA that encodes them.

As viral infection can change the activity of the BMP/SMAD signalling pathway it is advantageous to provide a compound that changes the activity or levels of intermediates in this pathway to return them to the levels in non-infected cells. This makes the environment disadvantageous for viral replication.

For example, HCV decreases the activity of the BMP/SMAD signalling pathway in such a way that the amount of hepcidin is decreased. It is advantageous to provide compounds that increase the activity of the BMP/SMAD signalling pathway in order to bring the amount or activity of the intermediates in the pathway to the levels at least of those in uninfected cells as this makes the environment in the cells unfavourable for viral replication and reduces replication of the virus.

For influenza virus infection, it is advantageous to provide compounds that increase the activity of the BMP/SMAD signalling pathway in order to bring the amount or activity of the intermediates in the pathway to the levels at least of those in uninfected cells or higher than in uninfected cells as this makes the environment in the cells unfavourable for influenza virus replication and reduces replication of influenza virus.

The compound may be formulated with any suitable excipient or carrier, for oral administration or for administration by intravenous or subcutaneous injection, or for intranasal administration, or for trans-cutaneous administration.

In one embodiment the compound may be BMP6 or an agonist of BMP6 signalling for use in the treatment or prevention of viral diseases, preferably BMP6 or an agonist of BMP6 for use in the treatment or prevention of HCV or hepatitis B virus (HBV).

In one embodiment the compound is not BMP7 for the treatment of HCV.

According to a further aspect the invention provides a method for obtaining an indication helpful in the assessment of whether viral infection in an individual will respond to treatment with antiviral treatment, comprising the steps of:
providing a sample of cells or a body fluid from the individual;
measuring the level in the cells or the body fluid of at least one indicator selected from: HAMP mRNA (which encodes hepcidin), ID1 mRNA, HJV mRNA, SMAD6 mRNA, and SMAD7 mRNA, hepcidin, hemojuvelin, SMAD6 protein, SMAD7 protein; and
comparing the level of the at least one indicator in the cells or the body fluid with the level of the same indicator in control cells or body fluid that not infected with the virus.

Preferably the method allows the determination of whether or not an individual is likely to respond to antiviral treatment.

In one embodiment the method may be helpful in the assessment of whether a liver-tropic virus, for example HCV or hepatitis B virus (HBV) in an individual will respond to antiviral treatment.

In one embodiment the method may be helpful in the assessment of whether HCV infection in an individual will respond to antiviral treatment with interferon and/or ribavirin. In this embodiment the sample of cells from the individual may be liver cells, for example cells taken from a liver biopsy.

In one embodiment of the method the body fluid may be blood and the at least one indicator may be hepcidin.

In the embodiment where the viral infection is HCV infection, cells from individuals that are less likely to respond to interferon and/or ribavirin treatment (non-responders (NR)) have reduced HAMP mRNA, reduced HJV mRNA and/or reduced levels of ID1 mRNA compared with control cells that are not infected with HCV.

Cells from non-responders also may have increased SMAD6 and/or increased SMAD7 compared to control cells that are not infected with HCV.

Cells from non-responders may also have altered ratios of these indicators. SMAD6 and SMAD7 may be relatively increased compared to ID1. SMAD7 may be relatively increased compared to HAMP, indicating inappropriately high expression of both I-SMADS (I-SMADS are SMAD6 and SMAD7).

The ratio of I-SMAD expression to HJV expression may also be significantly increased in cells from non-responders compared with cells that are not infected with HCV. HCV infected cells have reduced BMP signalling, correlating with suppressed hepcidin and non-response to conventional therapy. Therefore a decrease in indicators that correlate with increased hepcidin expression or an increase in indicators that correlate with decreased hepcidin expression in cells from an individual may indicate that the individual is a non-responder and/or may indicate that an individual will respond poorly to conventional anti-viral therapy, for example treatment with interferon and ribavirin.

Individuals who complete conventional antiviral treatment are classified as sustained virological responders (SVRs) if they were found to be HCV-RNA negative 6 months after treatment finished, or non-responders (NRs) if they remain HCV-RNA positive at the end of treatment. Treatment consists, for example of weekly Peg-IFN plus a daily dose of Ribavirin according to body weight.

According to a still further aspect the invention provides a kit for obtaining an indication useful in testing whether a viral infection in an individual will respond to treatment with an antiviral agent, the kit comprising a means for assessing the level in the cells of at least one indicator selected from: HAMP mRNA, HJV mRNA, ID1 mRNA, SMAD6 mRNA, and SMAD7 mRNA; and comparing the level of the at least one indicator in the cells with the level of the same indicator in control cells that are not infected with the virus.

Preferably the kit allows a determination to be made as to whether or not an individual will respond to treatment with an antiviral

The kit may be for use where the viral infection is a HCV infection and the antiviral agent is interferon and/or ribavirin.

The following examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Testing the Effect of BMP6 on Spread of Influenza A Virus Strain PR8 in HuH7 Hepatoma Cells In Vitro

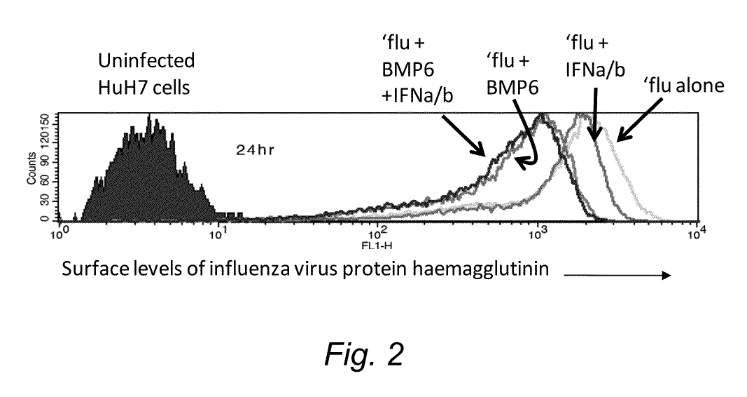

HuH7 cells were exposed to influenza virus at 10 plaque forming units per cell for one hour, and then cells were washed, and then either:
1) mixed with uninfected HuH7 cells in the ratio of 1:10, or
2) cultured together (all cells infected at start).
These two protocols were chosen as they would enable measurement of virus spread to uninfected cells or viral growth within infected cells, respectively. In both cases, cells were then left to grow and the virus to spread in the presence of recombinant IFNalpha and beta (100 Units/ml of each), 18 nM BMP6, or IFNs and BMP together. After 24 hr, cells were washed, and surface expression of the viral protein haemagglutinin was determined by monoclonal antibody staining and flow cytometry. Results of the experiment are shown in FIG. 2.

Effect of HCV Infection on the BMP/SMAD Signalling Pathway

Chronic HCV infection can lead to liver iron accumulation through suppressing the synthesis of the iron regulatory hormone, hepcidin. Production of hepcidin is stimulated by BMPs, and the iron overloading disorder hereditary haemochromatosis can be caused by defects in the BMP/SMAD signalling pathway that reduce hepcidin levels. Reduced hepcidin observed in HCV may be due to viral disruption of the BMP/SMAD signalling pathway.

Intermediates in the BMP/SMAD signalling pathway in liver were analysed in biopsies from patients and in cell culture models of HCV replication. It was surprisingly found that a) HCV suppressed BMP signalling at several different points along the BMP/SMAD signalling pathway; b) the changes in gene expression were similar in vivo and in vitro; and c) maximal disruption of the BMP/SMAD signalling pathway in biopsies correlated with non-response to antiviral therapy. The inhibition of BMP signalling is reminiscent of the disruption of IFN signalling by HCV that enables the virus to establish chronic infection. BMPs, like IFNs, may have antiviral activity. In an in vitro live virus model, it was found that BMP6 suppressed HCV growth over 5 days by over 90%.

Hepcidin maintains iron homeostasis. Regulation of hepcidin synthesis is complex but BMPs play an important role. Iron accumulation induces synthesis of BMP6 by the liver, which causes an increase in hepcidin expression through a signal transduction pathway involving the BMP co-receptor HJV and SMAD factors. Hepcidin then restricts dietary iron absorption and iron recycling through the blockade of the iron exporter ferroportin, returning the system to equilibrium. Persistently high levels of hepcidin reduce the iron flow to the erythron, causing anemia. Conversely, inappropriately low hepcidin underlies the iron overloading disorder hereditary hemochromatosis (HH). Chronic HCV infection is also associated with reduced hepcidin, and liver iron accumulation may occur, worsening inflammation and/or fibrosis. The suppressed hepcidin in the most common form of HH is thought to be due to disrupted BMP signalling; the expression of genes involved in the BMP/SMAD signalling pathway in HCV patients was therefore investigated (see Tables 1, 2)

TABLE 1
Clinical parameters of the control and HCV patients
Age, gender, and virological data relating to the control and HCV patients.
Where appropriate data is reported as the mean with the 95% confidence
interval.

| | Controls | HCV |
|---|---|---|
| | N = 8 | N = 57 |
| Age (yr) | 67.4 (53.9-81.0), | 44.1 (38.3-43.9), |
| | n = 7^{c} | n = 56^{c} |
| Gender (M/F) | 8/0 | 32/25 |
| Viral load (IU) | — | 1.30 × 10⁶ (7.46 × |
| | | 10⁵ − 1.85 × 10⁶), |
| | | n = 54^{c} |
| Viral genotype (⅓) | — | 42/15 |
| Fibrosis stage (0/mild/severe)^{a} | — | 35/22/0 |
| Treatment outcome (SVR/NR/ | — | 17/9/31 |
| other)^{b} | | |

^{a}Fibrosis data is reported as follows: 0 = no fibrosis; mild = METAVIR 1-2 and Ishak 1-3; severe = METAVIR 3-4 and Ishak 4-6.
^{b}Treatment outcome is reported as follows: SVR = HCV-RNA negative 6 months post completion of therapy; NR = HCV-RNA positive throughout therapy; other = outcome not yet known, lost on follow up or relapsed.
^{c}Missing patient information - data unavailable.

TABLE 2
Clinical parameters of the SVR and NR patients
Age, gender and virological data relating to the patients with known
treatment outcome. Where appropriate data is reported as the mean
with the 95% confidence interval.

| | SVR | NR |
|---|---|---|
| | N = 17 | N = 9 |
| Age (yr) | 36.8 (31.5-42.0), | 43.7 (37.3-50.1) |
| | n = 16^{b} | |
| Gender (M/F) | 9/8 | 4/5 |
| Viral load (IU) | 1.06 × 10⁶ (4.97 × 10⁵ − | 9.77 × 10⁵ (4.67 × 10⁴ − |
| | 1.61 × 10⁶), | 1.91 × 10⁶), |
| | n = 16^{b} | n = 8^{b} |
| Viral genotype (⅓) | 11/6 | 7/2 |
| Fibrosis grade | 10/7/0 | 3/6/0 |
| (0/mild/severe)^{a} | | |

^{a}Fibrosis data is reported as follows: 0 = no fibrosis; mild = METAVIR 1-2 and Ishak 1-3; severe = METAVIR 3-4 and Ishak 4-6.
^{b}Missing patient information - data unavailable.

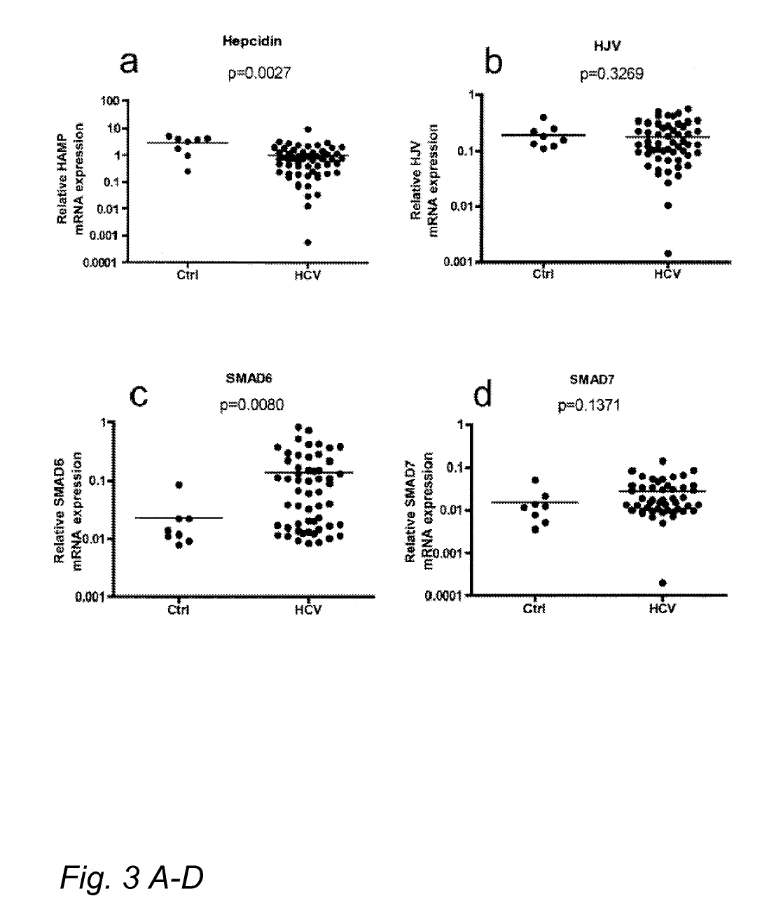

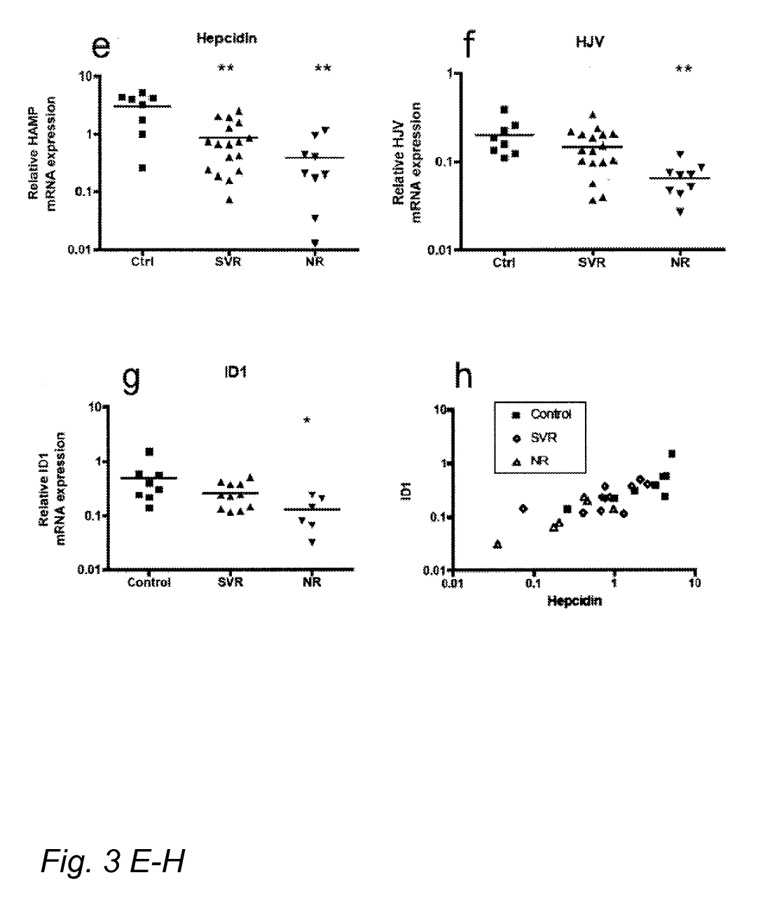

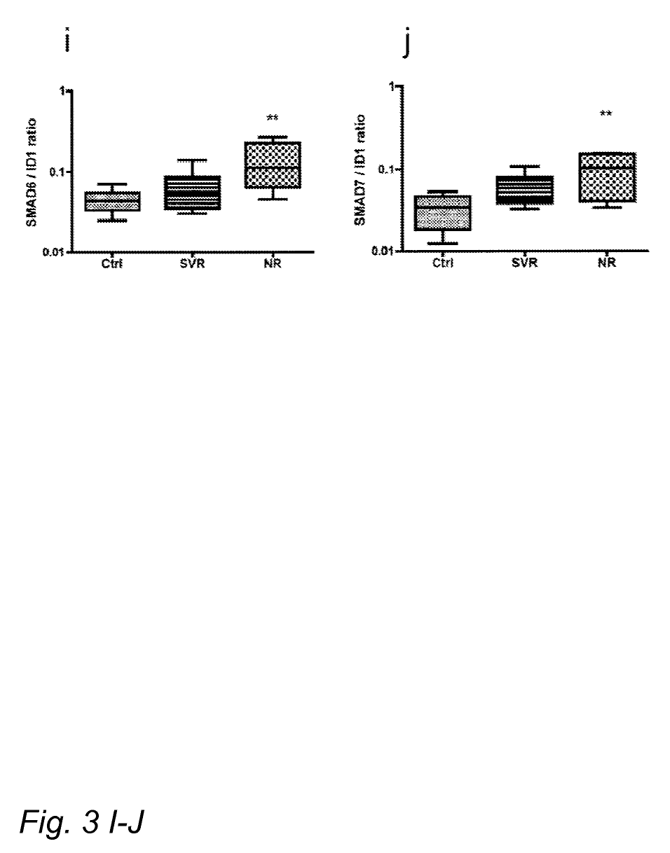

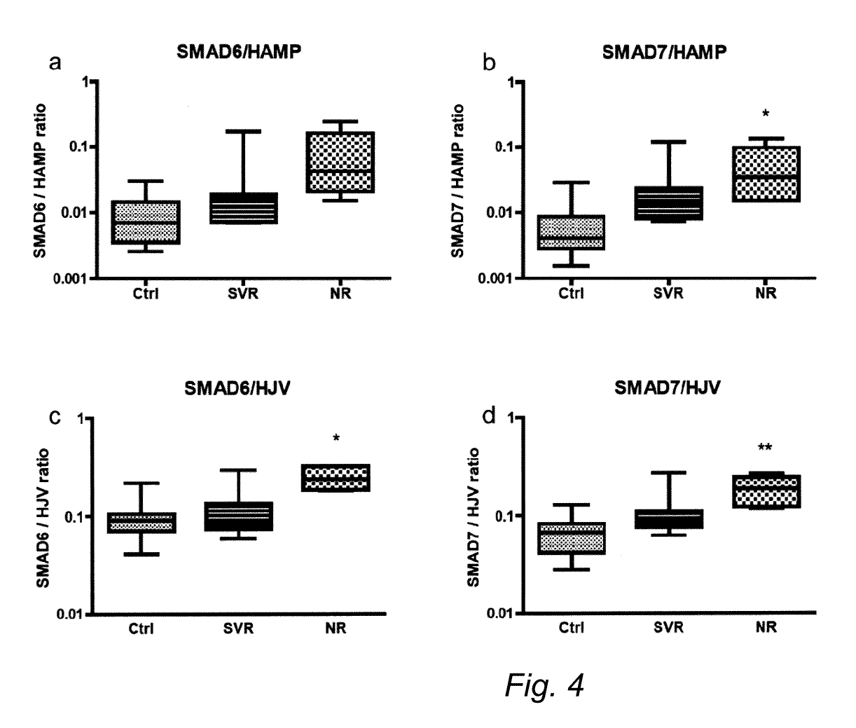

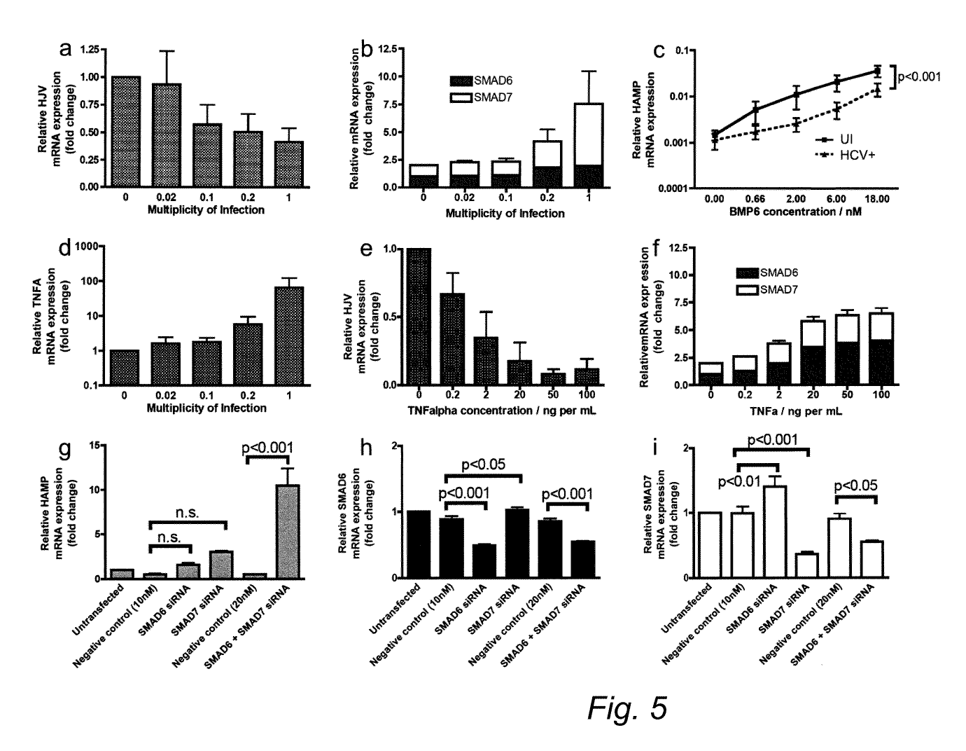

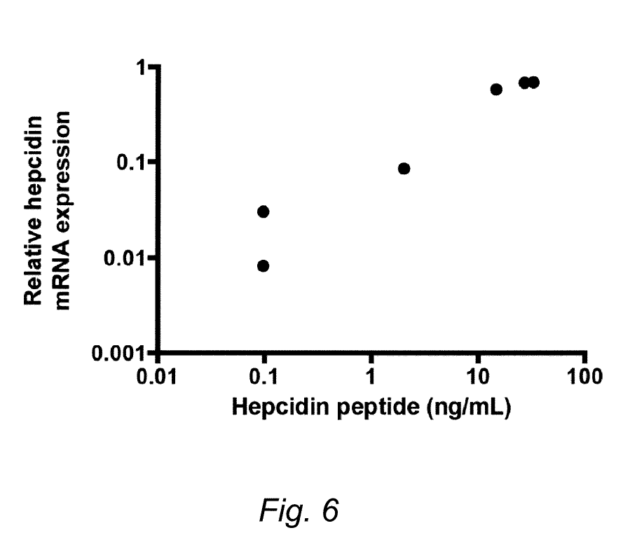

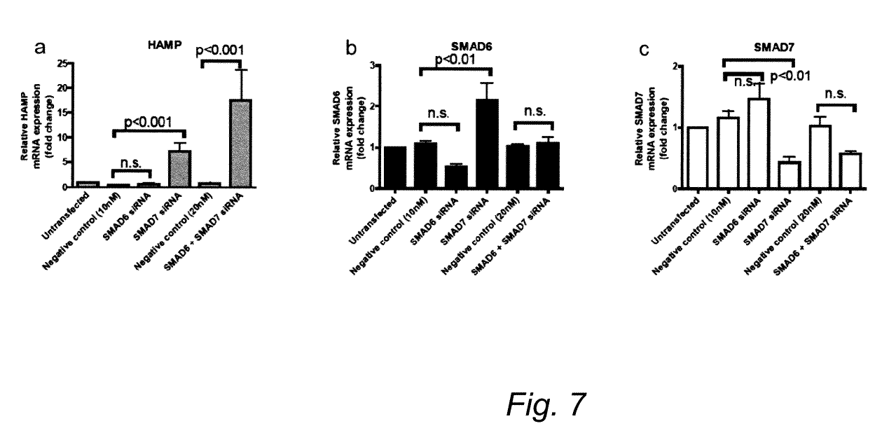

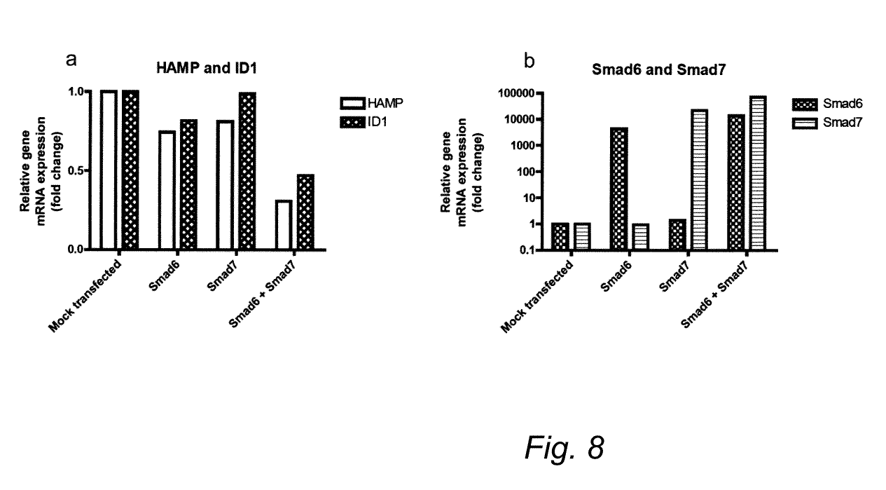

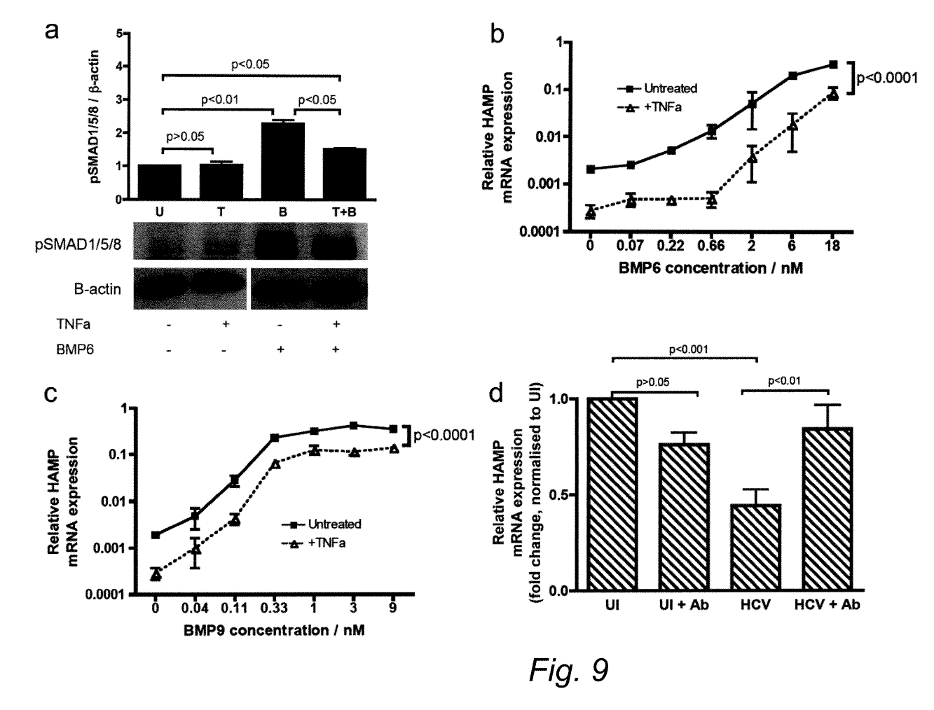

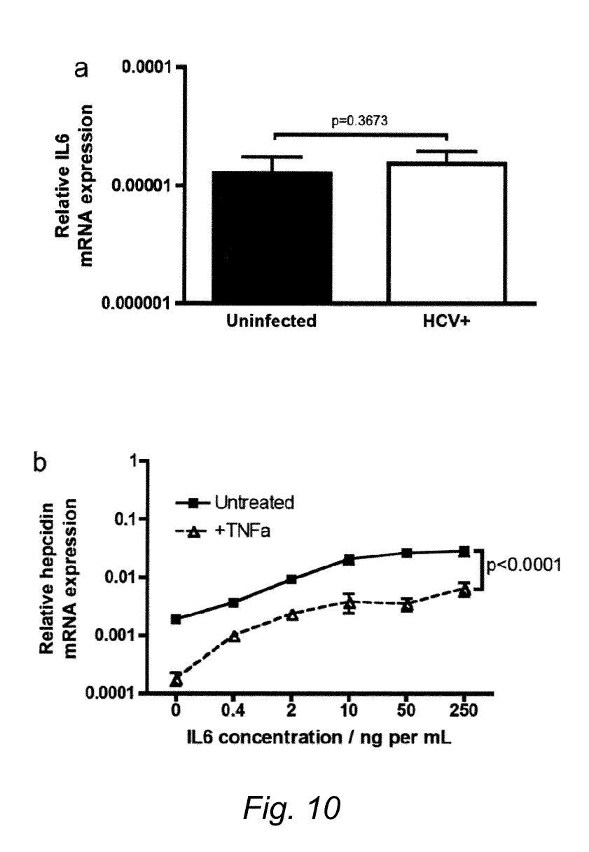

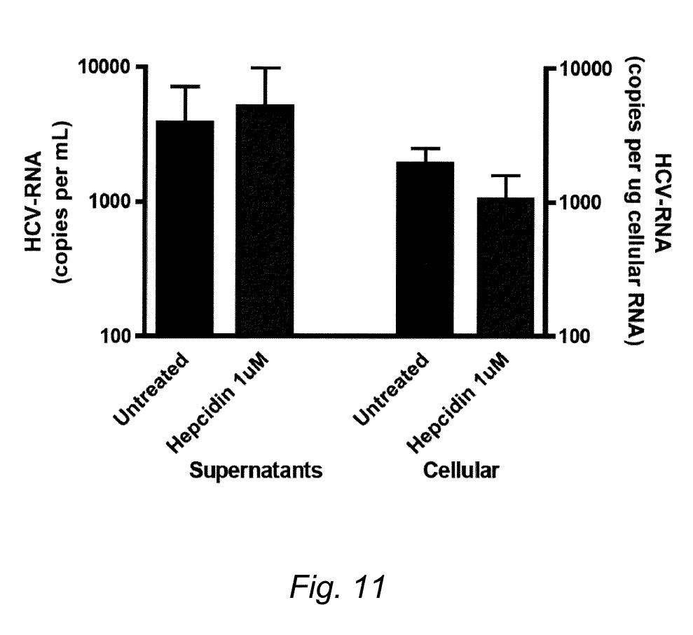

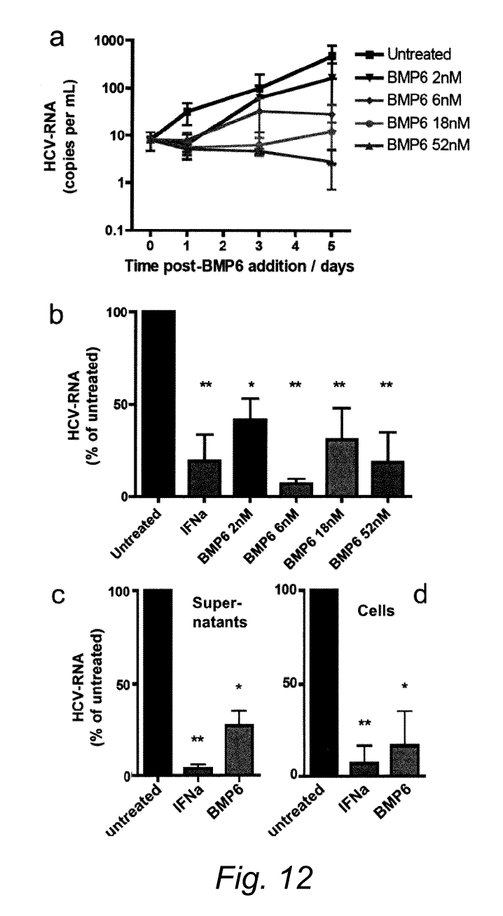

The expression of four genes was assessed: HAMP (which encodes hepcidin); the BMP co-receptor HJV, required for appropriate hepcidin synthesis; and SMAD6 and SMAD7, known as I-SMADs, which are switched on by BMP signalling but execute a negative feedback loop inhibiting SMAD-mediated signal transduction. Liver biopsies taken before antiviral treatment was commenced were assayed from a cohort of patients with HCV genotypes 1 or 3. FIGS. 3a-d show that, compared to mRNA levels in control liver tissue, biopsies from HCV patients had significantly reduced HAMP, and significantly increased SMAD6.

Because SMAD6 is generally considered to be an inhibitor of the BMP/SMAD signalling pathway, whereas SMAD7 inhibits both BMP and TGF-beta signalling, the data suggested an impairment of the BMP pathway in HCV infection. mRNA expression was analysed in pre-treatment biopsies from those patients whose eventual response to conventional antiviral therapy was known (sustained virological responder (SVR) or non-responder (NR)). NR had lower HAMP mRNA, reduced HJV mRNA and reduced levels of ID1, a canonical BMP target gene (FIG. 3e-g). Furthermore ID1 and HAMP levels correlated and were lowest in NR, consistent with reduced BMP signalling in this group (FIG. 3h). Both SMAD6 and SMAD7 are also BMP target genes; in the absence of other regulatory factors their levels should correlate with ID1. However in NRs, both SMAD6 and SMAD7 were relatively increased compared to ID1, and SMAD7 was relatively increased compared to HAMP, indicating inappropriately high expression of both I-SMADS (FIG. 3i, j and FIG. 4). The ratio of I-SMAD expression to HJV expression was also significantly increased in the NR group (FIG. 4). Together these findings indicate reduced BMP signalling in HCV patients, correlating with suppressed hepcidin and non-response to conventional therapy.

To investigate further, HuH7.5 hepatoma cells infected with replication-competent HCV were used (see Methods and Pietschmann, T. et al. Construction and characterization of infectious intragenotypic and intergenotypic hepatitis C virus chimeras. Proc Natl Acad Sci USA 103, 7408-7413 (2006)). First a full genome RNA-Seq data set that describes the effect of HCV infection on the transcriptome of HuH7.5 cells was interrogated for differences in genes in the BMP/SMAD signalling pathway (see methods in Woodhouse, S. D. et al. Transcriptome sequencing, microarray, and proteomic analyses reveal cellular and metabolic impact of hepatitis C virus infection in vitro. Hepatology 2010 August; 52(2):443-53. Table 3 shows that the expression of Type I and Type II BMP receptor genes and the protease TMPRSS6 that negatively regulates BMP signalling were not significantly altered in HCV infected cells, but the expression of HJV was reduced 2.6-fold and SMAD7 expression was significantly increased.

TABLE 3
RNA-seq data for genes in the BMP/SMAD signalling pathway from
HCV infected HuH7.5 cells, showing significant downregulation of HJV
and significant upregulation of SMAD7. Data derived from Woodhouse
et al (reference 20).

| | | Fold change on |
|---|---|---|
| Gene ID | Gene name | HCV infection |
| HFE2 (HJV) | Hemojuvelin | −2.6 |
| ACVRL1 (ALK1) | Activin A receptor type II-like 1 | −1.4 (n.s) |
| ACVR1 (ALK2) | Activin A receptor, type I | +1.3 (n.s) |
| BMPR1A (ALK3) | BMP receptor, type 1A | +1.3 (n.s) |
| BMPR1B (ALK6) | BMP receptor, type 1B | +1.8 (n.s) |
| ACVR2A | Activin A receptor, type IIA | 1.0 (n.s) |
| ACVR2B | Activin A receptor, type IIB | 1.0 (n.s) |
| BMPR2 | BMP receptor, type II | 1.0 (n.s) |
| ENG | Endoglin | +1.4 (n.s) |
| TMPRSS6 | Matriptase-2 | 1.0 (n.s) |
| SMAD7 | SMAD family member 7 | +1.9 |

n.s.—non-significant.

These expression patterns were confirmed in HuH7.5 cells exposed to a titration of multiplicities of infection, and showed that HJV decreased and both SMAD6 and SMAD7 increased as a function of increasing viral burden (FIG. 5a, b). These alterations suggested that virally infected cells might be relatively resistant to BMP-induced hepcidin expression. To test this, cells were exposed to recombinant BMP6 and HAMP mRNA was measured (HAMP mRNA detected by qRT-PCR correlated with ELISA-detected peptide—see FIG. 6). Virally infected cells exhibited a blunted hepcidin response compared to uninfected cells (FIG. 5c). The data indicate that the in vitro HCV infection model largely recapitulates the disruption of BMP signalling observed in liver biopsies.

In osteoblasts, BMP-induced differentiation is inhibited by TNF-alpha. Other studies have suggested TNF-alpha can suppress baseline hepcidin levels and HJV expression in liver-derived cells. Furthermore TNF-alpha is induced in HCV infection and higher pre-treatment levels may correlate with non-response to therapy. In our model system HCV infected cells upregulated TNFA mRNA levels (FIG. 5d). It was investigated how TNF-alpha affected the BMP/SMAD signalling pathway in uninfected hepatoma cells. Like HCV, TNF-alpha reduced HJV mRNA levels and increased both SMAD6 and SMAD7 mRNA (FIG. 5e, f). SMAD7 has been shown to suppress hepcidin expression, but the effect of SMAD6 or of the upregulation of both I-SMADs on hepcidin was unknown. First SMAD6 or SMAD7 or both genes were knocked-down. Knocking down either I-SMAD modestly increased hepcidin expression (FIG. 5g, FIG. 7). However we also observed that knock-down of one I-SMAD could induce higher mRNA levels of the other (FIG. 5h, i, FIG. 7). Circumventing this effect by knocking-down both I-SMADs led to a much higher upregulation of HAMP (FIG. 5g). This result suggests increases of both I-SMADs, as caused by HCV and TNF-alpha, would more potently suppress BMP signalling than if only one I-SMAD was increased. Consistent with this, overexpressing both murine I-Smads together in human hepatoma cells had the strongest effect in down-regulating HAMP and ID1 mRNA (FIG. 8).

Following BMP/BMP receptor binding, the signal cascade is conveyed by the phosphorylation of SMAD1/5/8. It was found that SMAD1/5/8 phosphorylation stimulated by 18 hr exposure to BMP6 was partially inhibited (˜35%) by TNF-alpha pre-treatment (FIG. 3a). TNF-alpha treatment severely blunted HAMP mRNA upregulation in response to both BMP6 (mean suppression 16.8-fold) and to a lesser extent the non-HJV binding BMP9 (4.3-fold) (FIG. 9b, c), suggesting that the suppressive effect of TNF-alpha operates by both reducing HJV expression and increasing I-SMADs. The above data demonstrate mechanistic similarities in how HCV and TNF-alpha suppress hepcidin, and so indicated that HCV disruption of BMP signalling may be caused by virally-induced TNF-alpha. To test this virally-infected cultures were incubated with neutralizing anti-TNF-alpha antibody. Anti-TNF-alpha significantly increased the hepcidin response to BMP6 in HCV-infected cells, restoring HAMP mRNA to levels found in uninfected cells (FIG. 9d).

The inhibition of BMP signalling by TNF-alpha is in marked contrast to the effect of interleukin-6 (IL-6)-synergy between IL-6 and BMPs induces high levels of hepcidin synthesis. In addition we observed that TNF-alpha treatment also blunted the hepcidin response to IL-6 (FIG. 10). In HCV infection, the balance within the liver of hepcidin-stimulatory signals (IL-6, BMPs) and hepcidin-antagonizing pathways (TNF-alpha, I-SMADs) is skewed towards the latter. This may explain the unusual lack of anemia accompanying chronic HCV infection compared to other persistent inflammatory states that are associated with increased hepcidin.

HCV interferes with the interferon (IFN) response, which may enable the development of chronic infection. Type I recombinant IFN is used as part of antiviral treatments to control infection, and recent findings show natural variations in the Type III IFN gene IL-28B correlate with response to treatment. By analogy, the reduced hepcidin and/or disruption of BMP signalling that has been observed might reflect an unsuspected role for these components in controlling HCV infection. No specific effect of hepcidin on HCV replication was detected (FIG. 11), suggesting a more general effect of BMP signalling on viral replication. To test this we added recombinant BMP6 immediately after infecting HuH7.5 cells with HCV. The subsequent accumulation of HCV RNA in culture supernatants over time was severely restricted by BMP6 (FIG. 12a). After 5 days BMP6 at all doses significantly reduced culture supernatant HCV RNA (FIG. 12b). Next BMP6 was added to HuH7.5 cultures in which HCV had already been replicating for 3 days. HCV RNA was measured after a further 7 days. Viral RNA in supernatants was reduced modestly by 2 nM and 6 nM BMP6 (not shown), and significantly by 18 nM BMP6 (FIG. 12c), and cellular HCV RNA was also significantly reduced by 18 nM BMP6 (FIG. 12d). These experiments show that stimulating the BMP signaling pathway reduces HCV replication in both newly infected cells and established virally-infected cultures.

In summary BMP signalling is identified as a target for HCV both in liver biopsies, in which disruption of the pathway correlates with non-response to antiviral therapy, and in vitro in cell culture, where BMP inhibition is mediated by virally induced TNF-alpha. Reversing this inhibition by increasing BMP signalling reduced HCV replication.

Methods

Patient Biopsy Samples

HCV patients were investigated who had presented at the following hospitals: Mater Misericordiae University Hospital, Dublin, Ireland (n=17); S. Bortolo Hospital, Vicenza, Italy (n=40); See tables 1 and 2 for further information. Liver biopsies were collected prior to the commencement of antiviral therapy using an 18-gauge needle and the sample split into two for both histological grading and gene expression analysis. Blood samples were obtained after an overnight fast from some patients for analysis of serum ferritin, transferrin saturation, iron, total iron binding capacity, full blood count and liver function tests including alanine aminotransferase. Informed consent was obtained from all patients and the study was approved by the relevant local ethics committees. All HCV patients were negative for HBV and HIV-1, and did not show clinical evidence of hemochromatosis (transferrin saturation<45%). Patients who had completed antiviral treatment were classified as sustained virological responders (SVRs) if they were found to be HCV-RNA negative 6 months after treatment finished, or non-responders (NRs) if they remained HCV-RNA positive throughout treatment. Treatment consisted of weekly Peg-IFN plus a daily dose of Ribavirin according to body weight. The mRNA from liver biopsies was extracted using RNeasy kits (Qiagen) and reverse transcribed using the High Capacity RNA-to-cDNA kit (Applied Biosystems). RNA was extracted with the inclusion of a gDNA elimination step from a subset of the biopsies (n=17) used to determine ID1 mRNA levels. Gene expression was assessed using qRT-PCR as described below, however a number of the 57 patient samples showed a lack of gene amplification for some genes, particularly where RNA was limited (HJV: n=2; SMAD6: n=2; SMAD7: n=9). Control liver biopsy mRNA samples were obtained from 3h Biomedical (Sweden) (all Caucasians, non-alcoholic, negative for viral hepatitis and haemochromatosis) and analysed alongside the HCV biopsy samples.

Quantitative Real-Time PCR (qRT-PCR)

RNA extraction and cDNA synthesis were carried out by using either RNeasy kits with QIAshredder homogenization (both Qiagen) and the High capacity RNA-to-cDNA kit (Applied Biosystems), or by using the Cells-to-Ct kit (Applied Biosystems), all according to the manufacturers' protocols. qRT-PCR reactions were performed on an Applied Biosystems Fast 7500 Real-Time PCR System (Applied Biosystems). Gene expression was assessed using inventoried Taqman Gene Expression Assays (Applied Biosystems) (see table 4 for assay codes) with Taqman Gene Expression Master Mix (Applied Biosystems) following the manufacturer's instructions. cDNA was diluted in Nuclease-Free Water (Ambion) to achieve a final concentration of 1-3 ng/uL. Samples were run in duplicate and gene expression levels were quantified relative to glyceraldehyde-3-phosphate dehydrogenase (GAPDH) mRNA expression using the delta Ct method; in some cases relative expression was then quantified further by normalizing to the untreated controls (delta-delta Ct method).

TABLE 4
Codes of the Taqman gene expression assays used in this study.

| Gene name | Assay ID |
|---|---|
| Bone morphogenetic protein 6 (BMP6) | Hs01099594_m1 |
| Glyceraldehyde-3-phosphate dehydrogenase | Hs99999905_m1 |
| (GAPDH) | |
| Hepcidin antimicrobial peptide (HAMP) | Hs00221783_m1 |
| Hemochromatosis type 2 (juvenile) (HJV) | Hs00377108_m1 |
| Inhibitor of DNA binding 1, dominant negative | Hs00357821_g1 |
| helix-loop-helix protein (ID1) | |
| SMAD family member 6 (SMAD6) | Hs00178579_m1 |
| SMAD family member 7 (SMAD7) | Hs00998193_m1 |
| Tumor necrosis factor (TNF superfamily, member 2) | Hs00174128_m1 |
| (TNF-alpha) | |
| MAD homolog 6 (Drosophila) (murine Smad6) | Mm01171378_m1 |
| MAD homolog 7 (Drosophila) (murine Smad7) | Mm00484742_m1 |

Hepatitis C Viral Infection

The Jc1 HCV strain was produced as described previously (Reference 20). Briefly, HuH7.5 cells were transfected with Jc1 RNA by electroporation and supernatants harvested 14-20 days post transfection. HuH7.5 cells were infected at a multiplicity of infection (MOI) of 0.02 unless otherwise stated. Infection was allowed to proceed for 9-11 days at which point infection was greater than 90% as determined by immunofluorescence (also described in Reference 20).

Cell Culture and Treatments

The hepatoma cell line Hep3B were maintained in MMEM supplemented with 10% foetal calf serum (PAA), 2 mM glutamine, 100 U/mL penicillin, 0.1 mg/mL streptomycin (all Sigma). HuH7 and HuH7.5 cells were cultured in DMEM supplemented as above. HuH7.5 cells infected at a MOI of 0.02 or hepatoma cells pre-treated for 48 h with TNF-alpha at 20 ng/mL were incubated overnight with titrations of human recombinant BMP6 or BMP9, and in the case of TNF-alpha treated cells IL-6 (all R&D Systems). Titrations of TNF-alpha were applied for 48 h prior to RNA extraction. A neutralizing anti-TNF-alpha antibody (clone 1825), (R&D Systems) supplemented the culture medium of HCV infected HuH7.5 cells at 0.2 μg/mL and was added at each sub culture from 2 days post infection to the end of the infection course. Uninfected cells were cultured and treated in parallel with the HCV infected cells.

sIRNA-Mediated Gene Knockdown

Hep3B or HuH7 cells were reverse-transfected with Silencer Select siRNA against SMAD6 and/or SMAD7 using siPORT NeoFX (all Ambion) following the manufacturer's recommended instructions for transfection in a 24 well plate. siRNA and siPORT NeoFX were diluted in Opti-MEM I medium (Invitrogen). Cells were assayed for gene expression 48 hours post transfection. siRNA s8411 and a custom siRNA (Sense: CCACAUUGUCUUACACUGA (SEQ ID NO: 1); Anti-sense: UCAGUGUAAGACAAUGUGG (SEQ ID NO: 2) were used to knock down SMAD6 and siRNAs s8412 and s8413 were used to target SMAD7. Each siRNA was used at 5 nM, and were premixed before transfection to yield siRNA mixtures at 10 nM where a single gene was targeted, or 20 nM where both I-SMADs were targeted. Cells were also transfected with a non-targeting siRNA (Negative Control Silencer Select part no. 4390843) (Ambion) at an equivalent concentration (10 nM or 20 nM) and with siRNA against GAPDH (Positive Control Silencer Select part no. 4390849) (Ambion).

Western Blot

Hep3B cells were exposed to TNF-alpha at 20 ng/mL for 48 hours before the addition of BMP6 at an end concentration of 2 nM. Lysates were harvested at either 1 h or 18 h post addition of BMP6. Briefly, cells were trypsinised and lysed for 20 min on ice in NP40 1% buffer supplemented with protease inhibitors (Sigma) at 1:500 and phosphatase inhibitor cocktail 2 (Sigma) at 1:100. The lysates were spun at 4° C., 13,000 rpm for 5 minutes and the supernatants stored at −80° C. until the blot was performed. Briefly, the protein content of the lysates was normalised using the BCA assay (Pierce) and run on 12% SDS-PAGE mini gels. Gels were blotted onto nitrocellulose membranes (GE Healthcare) and then blocked for 1 hour at room temperature in PBS containing 5% (w/v) milk. Membranes were then probed overnight at 4° C. in TBS-TWEEN containing 5% (w/v) BSA with the following primary antibodies: mouse anti-beta-actin (loading control) (Sigma), mouse anti-rabbit IgG (negative control) (Dako), rabbit anti-pSMAD1/5/8 (Cell Signalling), or rabbit anti-H.Pylori (negative control) (Dako). Membranes were washed and then incubated for 1 hour at room temperature with the relevant secondary antibodies: goat anti-mouse HRP (Dako) at 1:750 or donkey anti-rabbit HRP (Santa Cruz) at 1:10,000. Membranes were developed using ECL reagent (GE Healthcare), films were scanned using an AlphaScan (Alpha Innotech) running Epson Scan software (Seiko Epson), and band intensities were determined using ImageQuant5.2 (Molecular Dynamics).

Antiviral Experiments

5 day time course: cells infected as described above at a MOI of 0.02 for 2 h were plated and then immediately treated with BMP6 at the doses stated in the figures for the duration of the infection. Aliquots of supernatant were collected at the time points indicated in FIG. 12a. 10 day time course: cells infected for 2 h were plated and incubated for 3 days. Cells were sub-cultured and BMP6 added at the stated doses. Additional doses of the same concentration were applied at further sub-culturing to maintain the dose. For both time courses, IFNa (PBL Biomedical Laboratories) was added at 1000 U overnight either at day 4 (5 day time course) or day 9 (10 day time course). Supernatant RNA extraction was performed using QIAamp viral RNA extraction kit and total cellular RNA was extracted using RNeasy kit (both Qiagen). cDNA was then transcribed using the Superscript III Reverse transcriptase (Invitrogen) (random hexamers), all according to the manufacturers' protocols. HCV-RNA levels were measured using qRT-PCR in a LightCycler 480 Real-Time PCR System (Roche). cDNA at 10-100 ng/uL was amplified using RC1 (5′ GTC TAG CCA TGG CGT TAG TA 3′ (SEQ ID NO:3)) and RC21(5′ CTC CCG GGG CAC TCG CAA GC 3′ (SEQ ID NO:4)) primers. Each reaction was run in duplicate. HCV-RNA levels were quantified using a standard curve derived from HCV Jc1 cDNA.

Inhibitory SMAD Over-Expression Experiments

Plasmids encoding the murine orthologs of the inhibitory SMADs, Smad6 and Smad7, were a gift from C. Heldin (Uppsala University, Sweden). Plasmids were prepared using the Plasmid Maxi Prep Kit (Qiagen) following manufacturer's instructions and transfected into Hep3B cells using Effectene (Qiagen).

Hepcidin ELISA

To check that HAMP mRNA measurements correlated with secretion of hepcidin peptide, Hep3B cells were treated overnight with increasing concentrations of IL6 and BMP9 to induce varying amounts of hepcidin expression. HAMP mRNA was determined by qRT-PCR as described in Methods, and cell supernatant were analyzed for hepcidin peptide content using a Hepcidin ELISA kit (BaChem) as per the manufacturer's instructions. See FIG. 6.

Histone Deacetylase Activity Assay

Buffer A (10 mM HEPES, 0.2 mM EDTA, 1 mM EGTA, 10 mM KCl) and buffer C (20 mM HEPES, 1 mM EDTA, 10 mM EGTA, 400 mM NaCl), both pH 7.9, were pre-cooled and supplemented with DTT to 1 mM and protease inhibitors (Sigma). Cells were trypsinsed, centrifuged and washed in PBS. 1×10⁶ cells were then spun down and then resuspended in 400 μL of Buffer A and incubated on ice for 15 minutes. 25 μL of 10% NP-40 was then added followed by 10 s vortex and immediately afterwards a 1 minute spin at 13,000 rpm in a pre-cooled microfuge. The supernatant was removed and frozen (cytoplasmic extract) and the remaining pellet was resuspended in 50 μL of Buffer C. This suspension was then incubated on ice for a further 15 minutes with occasional vortexing before centrifugation at 13,000 rpm for 5 minutes in a pre-cooled microfuge. The supernatant from this spin was then removed and frozen (nuclear extract).

The HDAC activity of the cytoplasmic and nuclear extracts was then assayed using the Fluorometric HDAC Assay Kit (Enzo Life Sciences) following manufacturer's instructions. Briefly, 10-50 μg of protein per well (as determined by the Pierce BCA Protein Assay Kit (Thermo Scientific)) was diluted to constant volume with Buffer A for cytoplasmic extracts and Buffer C for nuclear extracts. Water alone was used for measuring a background reading, HeLa nuclear extract as a positive control and extract treated with Trichostatin A, a known HDAC inhibitor, acted as a negative control. The assay buffer and the assay substrate was then added to each well and incubated for 30 minutes at 37° C. followed by the addition of the developer reagent and a further 30 minute incubation at 37° C. Fluorescence was then assessed using a SpectraMax M2^{e} microplate reader and Soft Max Pro software (Molecular Devices, Silicon Valley, Calif., USA). The machine was set to excitation at 365 nm and emission at 470 nm. Each extract was assayed in triplicate, the background reading subtracted and then the value normalised to the total mass of protein present.

Data Analysis

Data was analysed using Microsoft Excel (Microsoft Inc.) and Graphpad Prism (Graphpad Software Inc.). Statistical analysis and graphical presentation of data was performed using Graphpad Prism. The statistical tests used are stated in figure legends; error bars denote the s.e.m.; p<0.05 was considered significant.

Where multiple data groups from the same experiment were analysed, a One-way ANOVA was performed with a Dunnett's post test to assess differences compared to the control. Where the distributions were not found to be Gaussian by the Kolmogorov-Smirnov test (where it was possible to perform the test) and the data sets were matched, the Friedman test was performed with the Dunn's post test to assess difference between data sets. Where data sets were matched the repeated measures 1-way ANOVA was performed with Bonferroni's Multiple Comparison Test to compute differences between pairs of data.

The paired t-test was performed where only two data sets were generated and subsequently compared from matched experiments. Where experiments were not matched the unpaired t-test was used. Both paired and unpaired t-tests were performed using the two-tailed distribution. Where the distribution was found not to be Gaussian by the Kolmogorov-Smirnov test (where it was possible to perform the test), the Mann-Whitney t-test was used.

It should be noted that the above description, attached materials and their descriptions are intended to be illustrative and not limiting of this invention. Many themes and variations of this invention will be suggested to one skilled in this and, in light of the disclosure. All such themes and variations are within the contemplation hereof. For instance, while this invention has been described in conjunction with the various exemplary embodiments outlined above, various alternatives, modifications, variations, improvements, and/or substantial equivalents, whether known or that rare or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments.

1. A method of treatment or prevention of a viral infection in a subject comprising administering to said subject an effective amount of a compound that is a modulator of the activity of at least one component of the BMP/SMAD signalling pathway.

2. The method according to claim 1 wherein the viral infection is an infection with a virus selected from hepatitis C virus (HCV), hepatitis B virus, influenza virus, HIV-1, HIV-2, respiratory syncytial virus (RSV) and vaccinia virus.

3. The method according to claim 1 wherein the viral infection is an infection with HCV or influenza virus.

4. The method according to claim 1 wherein the compound is an agonist of at least one component of the BMP/SMAD signalling pathway.

5. The method according to claim 1 wherein the compound is an antagonist of at least one component of the BMP/SMAD signalling pathway.

6. The method according to claim 1 wherein the compound increases the activity of the BMP/SMAD signalling pathway in the subject's cells.

7. The method according to claim 1 wherein the compound decreases the activity of the BMP/SMAD signalling pathway in the subject's cells.

8. The method according to claim 1 wherein the compound modulates the activity of the BMP/SMAD signalling pathway in the subject's cells towards the activity expected in non virus infected cells.

9. The method according to claim 1 wherein the compound increases the activity of the BMP/SMAD signalling pathway in the subject's cells to a higher activity than expected in non virus-infected cells.

10. The method according to claim 1 wherein the compound decreases the activity of the BMP/SMAD signalling pathway in the subject's cells to a lower activity than expected in non virus-infected cells.

11. A method for identifying a compound that is useful in the treatment of infection with a virus comprising the steps of:
selecting a compound that modulates the activity of an intermediate in the BMP/SMAD signalling pathway;
testing whether the compound reduces or prevents replication of said virus in virus-infected cells in vitro.

12. The method according to claim 11, further comprising the step of making a quantity of the selected compound.

13. The method according to claim 11, wherein the virus is HCV or influenza virus.

14. A method for obtaining an indication helpful in the assessment of whether viral infection in an individual will respond to treatment with antiviral treatment, comprising the steps of:
providing a sample of cells or a body fluid from the individual;
measuring the level in the cells or the body fluid of at least one indicator selected from: HAMP mRNA (which encodes hepcidin), ID1 mRNA, HJV mRNA, SMAD6 mRNA, and SMAD7 mRNA, hepcidin, hemojuvelin, SMAD6 protein, SMAD7 protein; and comparing the level of the at least one indicator in the cells or the body fluid with the level of the same indicator expected in control cells or body fluid that is not infected with the virus.

15. The method according to claim 14 wherein the viral infection is HCV infection and the cells have reduced HAMP mRNA, reduced HJV mRNA and/or reduced levels of ID1 mRNA compared with control cells that are not infected with HCV.

16. The method according to claim 14 wherein the infection is with HCV and the cells have increased SMAD6 and/or increased SMAD7 compared to control cells that are not infected with HCV.

17. The method according to claim 14 wherein the infection is an infection with HCV and SMAD6 and SMAD7 are relatively increased compared to ID1 in the cells and/or SMAD7 is relatively increased compared to HAMP in the cells.

18. The method according to claim 14 wherein the sample is a sample of liver cells and the virus is HCV.

19. The method according to claim 14 wherein the virus is HCV and the antiviral treatment is treatment with interferon and/or ribavirin.

20. The method according to claim 14 wherein the body fluid is blood and the at least one indicator is hepcidin.

21. A method for inhibiting viral replication comprising modulating the activity of a component of the BMP/SMAD signalling pathway.

22. A method according to claim 21 wherein the viral replication is replication of HCV or influenza virus.


SEQUENCE LISTING (4 sequences)

SEQ ID NO: 1 - RNA, 19 nt, Artificial Sequence
  Custom siRNA
ccacauugucuuacacuga

SEQ ID NO: 2 - RNA, 19 nt, Artificial Sequence
  Custom siRNA
ucaguguaagacaaugugg

SEQ ID NO: 3 - DNA, 20 bp, Artificial Sequence
  Synthetic primer
gtctagccatggcgttagta

SEQ ID NO: 4 - DNA, 20 bp, Artificial Sequence
  Synthetic primer
ctcccggggcactcgcaagc